(19) United States
(12) Patent Application Publication
(10) Pub. No.: US 2016/0244391 A1
(43) Pub. Date: Aug. 25, 2016

(54) CATALYST AND PROCESS FOR PRODUCING ALDEHYDES AND/OR ALCOHOLS

(71) Applicant: DAICEL CORPORATION, Osaka (JP)

(72) Inventors: Tetsu NAKATANI, Himeji-shi (JP); Hiroyuki FUJII, Himeji-shi (JP); Yasutaka TANAKA, Himeji-shi (JP); Jun NAKANISHI, Himeji-shi (JP)

(73) Assignee: DAICEL CORPORATION, Osaka (JP)

(21) Appl. No.: 15/019,583

(22) Filed: Feb. 9, 2016

(30) Foreign Application Priority Data
Feb. 19, 2015 (JP) 2015-030642

(51) Int. Cl. C07C 45/41; C07C 29/149; B01J 37/08; B01J 23/89; B01J 37/02

(52) U.S. Cl. CPC C07C 45/41; B01J 23/8993; B01J 37/0236; B01J 37/08; C07C 29/149

(57) ABSTRACT

A catalyst includes molybdenum, palladium, and an iron oxide, for producing at least one of an aldehyde and an alcohol from a carboxylic acid via hydrogenation. The catalyst preferably contains 0.1 to 40 parts by weight of molybdenum and 1 to 50 parts by weight of palladium per 100 parts by weight of the iron oxide. The catalyst is preferably obtained by preparing a dispersion or solution containing a molybdenum component, a palladium component, and an iron component, drying the dispersion or solution to give a solid, and firing the solid.

### TECHNICAL FIELD

The present invention relates to catalysts and processes for producing aldehydes and/or alcohols. More specifically, the present invention relates to a catalyst for producing an aldehyde and/or an alcohol from a carboxylic acid; and to a process for producing an aldehyde and/or an alcohol from a carboxylic acid via hydrogenation using the catalyst.

### BACKGROUND ART

Japanese Unexamined Patent Application Publication (JP-A) No. H11-322658 (PTL 1) discloses a catalyst for producing acetaldehyde from acetic acid via hydrogenation. This catalyst is an iron oxide catalyst (palladium-supporting iron oxide catalyst) containing 2.5% to 90% by weight of palladium. This literature also discloses a method for producing acetaldehyde from acetic acid using the catalyst and discloses that the method gives a gaseous product containing, in addition to main product acetaldehyde, other components such as methane, ethane, ethylene, carbon dioxide, acetone, ethanol, ethyl acetate, water, and unreacted acetic acid.

Japanese Unexamined Patent Application Publication (Translation of PCT Application) (JP-A) No. 2011-529494 (PTL 2) discloses a process for producing acetaldehyde from acetic acid via hydrogenation using a catalyst, where the catalyst includes iron, ruthenium, platinum, and/or tin on a silica support.

JP-A No. 2012-153698 (PTL 3) discloses a process for producing acetaldehyde from acetic acid via hydrogenation using a catalyst, where the catalyst includes cobalt and at least one of iron and molybdenum, supported on an iron oxide or silica support.

JOURNAL OF CATALYSIS 168, 255-264 (1997) (NPL 1) discloses a process for producing acetaldehyde from acetic acid via hydrogenation using a platinum-supporting iron oxide catalyst and discloses that the process gives acetaldehyde highly selectively.

### CITATION LIST

### Patent Literature

PTL 1: JP-A No. H11-322658
PTL 2: JP-A No. 2011-529494
PTL 3: JP-A No. 2012-153698

### Non Patent Literature

NPL 1: JOURNAL OF CATALYSIS 168, 255-264 (1997)

### SUMMARY OF INVENTION

### Technical Problem

The inventors of the present invention found that a catalyst including an iron component as in the literature, when used in hydrogenation reactions disadvantageously suffer from coking. Specifically, carboxylic acids (such as acetic acid) are decomposed, via iron carbides, into carbon, and the carbon is deposited on the catalyst in the “coking”. The coking causes the catalyst, when pelletized, to collapse in shape and/or causes the catalyst to have lower strength. Examples of possible solutions so as to restrain the coking include a technique of adding CO₂ and/or H₂O to the reaction gas and allowing this to react with deposited carbon; and a technique of increasing a hydrogen to carbon ratio in the reaction gas. The inventors, however, found that these techniques cause the reactivity to decrease and fail to achieve target reaction performance such as selectivity and yield.

Accordingly, the present invention has an object to provide a catalyst for producing aldehydes and/or alcohols from carboxylic acids via hydrogenation, where the catalyst less suffers from coking upon the production and gives the aldehydes and/or alcohols with excellent selectivity in excellent yields. The present invention has another object to provide a process for producing aldehydes and/or alcohols from carboxylic acids using the catalyst, where the process gives the aldehydes and/or alcohols with excellent selectivity in excellent yields.

### Solution to Problem

After intensive investigations to achieve the objects, the inventors have found that a catalyst including molybdenum, palladium, and an iron oxide, when used for producing an aldehyde and/or an alcohol from a carboxylic acid via hydrogenation, less suffer from coking and gives the aldehydes and/or alcohols with excellent selectivity in excellent yields. The present invention has been made based on these findings.

Specifically, the present invention provides, according to an embodiment, a catalyst for producing at least one of an aldehyde and an alcohol from a carboxylic acid via hydrogenation. The catalyst includes molybdenum, palladium, and an iron oxide.

The catalyst according to the embodiment of the present invention preferably contains 0.1 to 40 parts by weight of molybdenum and 1 to 50 parts by weight of palladium per 100 parts by weight of the iron oxide.

The catalyst according to the embodiment of the present invention is preferably obtained by preparing a dispersion or solution containing a molybdenum component, a palladium component, and an iron component, drying the dispersion or solution to give a solid, and firing the solid.

The catalyst according to the embodiment of the present invention is preferably in the form of pellets.

The present invention also provides, according to another embodiment, a process for producing at least one of an aldehyde and an alcohol. The process produces the at least one of an aldehyde and an alcohol by hydrogenating a carboxylic acid in a gas phase in the presence of the catalyst.

The process according to the embodiment of the present invention for producing an aldehyde and/or an alcohol preferably hydrogenates acetic acid to give at least one of acetaldehyde and ethanol.

### Advantageous Effects of Invention

With catalyst according to the embodiment of the present invention, coking is restrained upon production of aldehydes and/or alcohols from carboxylic acids via hydrogenation. This protects the catalyst from collapsing, when pelletized, and from strength deterioration. With the process according to the embodiment of the present invention for producing an aldehyde and/or an alcohol, by-production of hydrocarbons having two or more carbon atoms are restrained, where the hydrocarbons are formed upon production of the aldehyde and/or alcohol from a carboxylic acid via hydrogenation. In addition, control of reaction conditions in the process restrains formation of other by-products such as ketones and carbon dioxide, where the other by-products are formed by other side reactions. This configuration leads to selective formation of hydrogenation products (aldehydes and alcohols).

### BRIEF DESCRIPTION OF DRAWINGS

FIG. 1 is a schematic flow diagram illustrating a process according to an embodiment of the present invention for producing an aldehyde and/or an alcohol.

### DESCRIPTION OF EMBODIMENTS

Catalyst

The catalyst according to the embodiment of the present invention is a catalyst for producing an aldehyde and/or an alcohol from a carboxylic acid via hydrogenation (catalyst for aldehyde and/or alcohol production) and includes molybdenum, palladium, and an iron oxide. The “iron oxide” refers to and includes not only Fe₂O₃, but also FeO, Fe₃O₄, and other oxides in other oxidation states. The term “(an) aldehyde and/or (an) alcohol” refers to either one or both of an aldehyde and an alcohol. Hereinafter “(an) aldehyde and/or (an) alcohol” is also simply referred to as a “target product(s)”.

The catalyst according to the embodiment of the present invention may contain the iron oxide in a content of typically 30% by weight or more (30% to 99% by weight), preferably 40% to 98% by weight, more preferably 50% to 97% by weight, furthermore preferably 60% to 96% by weight, and particularly preferably 70% to 95% by weight, based on the total amount of the catalyst. The catalyst, when containing the iron oxide in a content within the range, maintains sufficient catalytic activity and offers high selectivity to the target product(s). The content of the iron oxide may be calculated by performing elementary analysis of the catalyst to determine the content of iron (Fe) element, and converting the content in terms of Fe₂O₃.

The proportion of molybdenum (proportion in terms of molybdenum (Mo) element) in the catalyst according to the embodiment of the present invention is typically 0.1 to 40 parts by weight, preferably 0.3 to 38 parts by weight, more preferably 0.5 to 36 parts by weight, furthermore preferably 0.7 to 34 parts by weight, and particularly preferably 1.0 to 30 parts by weight, per 100 parts by weight of the iron oxide. The content of molybdenum (content in terms of molybdenum element) based on the total amount of the catalyst is typically 0.07% to 28% by weight, preferably 0.2% to 27% by weight, more preferably 0.4% to 26% by weight, furthermore preferably 0.5% to 25% by weight, and particularly preferably 0.8% to 23% by weight. The catalyst, when containing molybdenum in a proportion (content) within the range, less suffers from coking and thereby less suffers from collapse of the catalyst pellets and deterioration in strength. The molybdenum proportion (content) may be calculated by performing elementary analysis of the catalyst to determine the content of molybdenum (Mo) element and calculating the proportion from the content.

The proportion of palladium (proportion in terms of palladium element) in the catalyst according to the embodiment of the present invention is typically 1 to 50 parts by weight, preferably 1.4 to 40 parts by weight, more preferably 1.6 to 35 parts by weight, furthermore preferably 1.8 to 30 parts by weight, and particularly preferably 2.0 to 25 parts by weight, per 100 parts by weight of the iron oxide. The content of palladium (content in terms of palladium element) based on the total amount of the catalyst is typically 0.7% to 33% by weight, preferably 1.0% to 29% by weight, more preferably 1.2% to 26% by weight, furthermore preferably 1.3% to 23% by weight, and particularly preferably 1.5% to 20% by weight. The catalyst, when containing palladium in a proportion (content) within the range, maintains sufficient catalytic activity and offers high selectivity to the target product(s). After firing, palladium is generally present also as oxides such as PdO. These oxides, however, are reduced to metal palladium (Pd) in a reducing atmosphere (e.g., in a H₂ atmosphere) upon production of the target product(s) from a carboxylic acid via hydrogenation. The palladium proportion (content) may be determined by performing elementary analysis of the catalyst to determine the content of palladium (Pd) element, and calculating the proportion from the content.

The catalyst according to the embodiment of the present invention contains, per 100 parts by weight of the iron oxide, preferably molybdenum in a proportion (proportion in terms of molybdenum element) of 0.1 to 40 parts by weight and palladium in a proportion (proportion in terms of palladium element) of 1 to 50 parts by weight; and more preferably molybdenum in a proportion (proportion in terms of molybdenum element) of 0.3 to 38 parts by weight and palladium in a proportion (proportion in terms of palladium element) of 1.4 to 40 parts by weight. The catalyst, when containing these components in proportions (contents) within the ranges, maintains sufficient catalytic activity, less suffers from coking, less suffers from collapse of the catalyst pellets and deterioration in strength, and offers high selectivity to the target product(s).

The catalyst according to the embodiment of the present invention may be coexistent with a support typically of silica, and/or may further contain a metal oxide within ranges not adversely affecting advantageous effects of the present invention. Examples of the metal oxide include, but are not limited to, germanium oxides, tin oxides, vanadium oxides, and zinc oxide. The catalyst may contain one or more of these metal oxides combined with one or more other metals such as platinum, copper, and gold. The content of the metal oxide (excluding iron oxides) is typically about 0.1% to about 50% by weight based on the total amount of the catalyst according to the embodiment of the present invention. The amount of the other metal (excluding molybdenum and palladium) to be added is typically about 0.1% to about 50% by weight based on the total amount of the catalyst according to the embodiment of the present invention.

The catalyst according to the embodiment of the present invention may be in any form not limited, but is preferably in the form of pellets. This is because such pellet-form catalyst readily maintains its catalytic activity at sufficient levels, is easy to handle, and is easy to use in the reaction. The “pellet-form catalyst” refers to a catalyst in the form of particles of a size of about 1 to about 10 mm. This may be cylindrical particles having a cylindrical diameter of about 1 to about 10 mm and a height (length) of about 1 to about 10 mm. Assume that an intermediate product aldehyde in the carboxylic acid hydrogenation is to be selectively obtained. In particular in this case, the catalyst components are preferably supported approximately only on surfaces of inert support pellets so as to eliminate or minimize the progress of consecutive reactions in pores.

The catalyst according to the embodiment of the present invention effectively restrains coking, i.e., has a significantly reduced amount of carbon deposited on the catalyst after the reaction, as compared typically with the catalyst disclosed in PTL 1. The catalyst according to the embodiment of the present invention may have an increase of the amount of carbon deposited on the catalyst after the reaction of 5% by weight or less, and preferably 3% by weight or less, as compared with the deposited carbon amount before the reaction. The catalyst, when having an increase in the deposited carbon amount of 5% by weight or less, less suffers from collapse of the catalyst pellets and deterioration in strength. The carbon amount may be measured typically with any of X-ray fluorescence analyzers and organic elemental analyzers. Examples of conditions for the reaction include, but are not limited to, conditions for catalyst property evaluation described in working examples.

Advantageously, the catalyst according to the embodiment of the present invention less suffers from collapse of the catalyst pellets and deterioration in strength as compared typically with the catalyst disclosed in PTL 1. For example, assume that the catalyst according to the embodiment of the present invention is in the form of cylindrical pellets having a diameter of 5 mm and a height (length) of 5 mm. In this case, the catalyst has a crushing strength after the reaction of typically 50 N or more, preferably 75 N or more, and more preferably 100 N or more. The crushing strength may be determined typically by applying force to the flat portion of a sample cylindrical pellet having a diameter of 5 mm and a height of 5 mm using the digital hardness meter KHT-40N (Fujiwara Scientific Co., Ltd.) and measuring the strength at the time when the pellet collapses. Examples of conditions for the reaction herein include, but are not limited to, conditions for catalyst property evaluation described in the working examples.

The catalyst according to the embodiment of the present invention is preferably obtained by preparing a dispersion or solution containing a molybdenum component (component containing the molybdenum), a palladium component (component containing palladium), and an iron component, drying the dispersion or solution to give a solid, and firing the solid. Specifically, the catalyst according to the embodiment of the present invention may be prepared typically by a method including steps (1), (2), (3), and (4) as follows.

In the step (1), the molybdenum component, the palladium component, and the iron component, as well as other necessary components and a solvent are combined and stirred to give a dispersion or solution.

In the step (2), the dispersion or solution is heated to evaporate to dryness.

In the step (3), the article after evaporation to dryness is dried.

In the step (4), the article after drying is fired. The method including the steps (1), (2), (3), and (4) gives oxides as a powdery catalyst. The iron component is converted into an oxide, i.e., an iron oxide, after firing. Namely, the iron component is a precursor component that is converted into an iron oxide via firing. The step (2) of evaporating to dryness and the step (3) of drying may be performed not separately, but at once.

Instead of the preparation method, the catalyst according to the embodiment of the present invention may be prepared typically by a method of supporting components such as the iron component, molybdenum component, and palladium component onto a support such as silicon dioxide (silica) or alumina. Specifically, the catalyst according to the embodiment of the present invention may be prepared by a method in which a solution or dispersion containing components such as the iron component, molybdenum component, and palladium component is prepared; a support such as silica or alumina is immersed in the solution or dispersion and thereby impregnated with the components such as the iron component, molybdenum component, and palladium component. The resulting article including the solution or dispersion is evaporated to dryness to give a catalyst, and the catalyst is dried under reduced pressure or at normal atmospheric pressure, and the dried catalyst is fired. This preparation method gives oxides as a powdery catalyst. The iron component is converted into an oxide, i.e., an iron oxide, after firing. The impregnation with the components such as the iron component, molybdenum component, and palladium component may be performed according to a known or common procedure.

The amount of the support such as silicon dioxide (silica) or alumina is typically 10 to 500 parts by weight, and preferably 30 to 300 parts by weight, per 100 parts by weight of the total amount of metallic compounds to be used. The support such as silicon dioxide (silica) or alumina may be selected from commercially available products. Typically, a non-limiting example of silicon dioxide (silica) is Aerosil 200 available from Nippon Aerosil Co., Ltd. Assume that an intermediate product aldehyde in the carboxylic acid hydrogenation is to be selectively obtained. In particular in this case, the support is preferably one that is approximately devoid of pores or has, if any, pores having a pore size of 0.1 μm or more. This is preferred to eliminate or minimize the progress of consecutive reactions in the pores.

The molybdenum component is not limited, as long as being a compound (molybdenum compound) containing molybdenum (Mo) element, and is exemplified by, but not limited to, ammonium molybdates such as hexaammonium molybdate tetrahydrate; and molybdenum oxides. The blending amount of the molybdenum component is typically 0.5% to 50% by weight, and preferably 1.0% to 30% by weight, based on the total amount (100% by weight) of metallic compounds to be used. The molybdenum component may also be selected from commercially available products. Each of different molybdenum components may be used alone or in combination.

The palladium component is not limited, as long as being a compound (palladium compound) containing palladium (Pd) element, and is exemplified by palladium nitrates such as palladium(II) nitrate hydrate; palladium sulfate; palladium chloride; palladium oxide; and palladium acetate. The blending amount of the palladium component is typically 0.5% to 50% by weight, and preferably 1.0% to 30% by weight based on the total amount (100% by weight) of metallic compounds to be used. The palladium component may also be selected from commercially available products. Each of different palladium components may be used alone or in combination.

The iron component is not limited, as long as being a compound (iron compound) containing iron (Fe) element, and is exemplified by oxides such as iron oxides; nitrides such as iron nitrides; and other iron compounds. Examples of the other iron compounds include, but are not limited to, iron nitrates such as iron(III) nitrate hexahydrate; iron chlorides such as iron(III) chloride hexahydrate; and iron sulfates. The amount of the iron component to be blended is typically 20% to 99% by weight, and preferably 40% to 95% by weight, based on the total amount (100% by weight) of metallic compounds to be used. The iron component may also be selected from commercially available products. Each of different iron components may be used alone or in combination. Assume that iron sulfate is used as the iron component. In this case, sulfate ions should be removed from the iron component by adding an alkaline precipitant such as ammonia to the iron sulfate to give precipitates as an insoluble iron compound, and sufficiently washing the precipitates with water.

Examples of the solvent include, but are not limited to, water, alcohols, and toluene, of which water is preferred. Accordingly, the dispersion or solution is preferably an aqueous solution or aqueous dispersion. The amount of the solvent is not limited, as long as capable of dispersing or dissolving the metallic compounds (components), but is typically 100 to 5000 parts by weight, and preferably 300 to 1000 parts by weight, per 100 parts by weight of the total amount of the metallic compounds to be used. The coexistence of a chelating agent in the dispersion or solution is also effective for better catalytic activity. This is because salts of platinum-group metals such as palladium more readily precipitate as compared with salts of base metals such as iron. Examples of the chelating agent include, but are not limited to, citric acid and ethylenediaminetetraacetic acid (EDTA). The amount of the chelating agent is typically 10 to 1000 parts by weight per 100 parts by weight of the solvent.

The evaporation to dryness may be performed typically at a temperature of 50° C. to 150° C. for 3 to 48 hours. The drying may be performed typically at a temperature of 50° C. to 300° C. for 1 to 48 hours. The firing may be performed typically at a temperature of 200° C. to 600° C. for 1 to 24 hours. The evaporation to dryness, drying, and firing may be performed in an air atmosphere typically with a common electric furnace. After firing, the resulting powdery catalyst may be further subjected to processing or treatment. For example, the powdery catalyst may be compressed into tablets, be compacted and shaped into pellets, be crushed, and/or be classified typically with a mesh.

Aldehyde and/or Alcohol Production Process

The process according to the embodiment of the present invention produces an aldehyde and/or an alcohol from a carboxylic acid via hydrogenation in a gas phase in the presence of the catalyst according to the embodiment of the present invention. Hereinafter the process according to the embodiment of the present invention for producing an aldehyde and/or an alcohol is also simply referred to as the production process according to the embodiment of the present invention. The production process according to the embodiment of the present invention may produce an aldehyde alone, an alcohol alone, or both an aldehyde and an alcohol. In particular, the production process according to the embodiment of the present invention preferably produces at least an aldehyde. The hydrogenation in the production process according to the embodiment of the present invention is preferably performed using hydrogen (H₂) gas.

The “carboxylic acid” refers to an organic acid containing at least one carboxy group per molecule. Examples of the carboxylic acid include, but are not limited to, formic acid, acetic acid, propionic acid, butyric acid, valeric acid, acrylic acid, and benzoic acid.

The “aldehyde” refers to a hydrocarbon compound containing at least one formyl group per molecule. Examples of the aldehyde include, but are not limited to, formaldehyde, acetaldehyde, propionaldehyde, butanal, pentanal, acrolein, and benzaldehyde. The production process according to the embodiment of the present invention gives an aldehyde corresponding to the starting material carboxylic acid.

The “alcohol” refers to a hydrocarbon compound containing at least one hydroxy group per molecule. Examples of the alcohol include, but are not limited to, methanol, ethanol, propanol, butanol, isopropyl alcohol, pentanol, and ethylene glycol. The production process according to the embodiment of the present invention gives an alcohol corresponding to the starting material carboxylic acid.

In the production process according to the embodiment of the present invention, it is preferred that the carboxylic acid is acetic acid, the aldehyde is acetaldehyde, and the alcohol is ethanol. Namely, the process preferably produces acetaldehyde and/or ethanol from acetic acid.

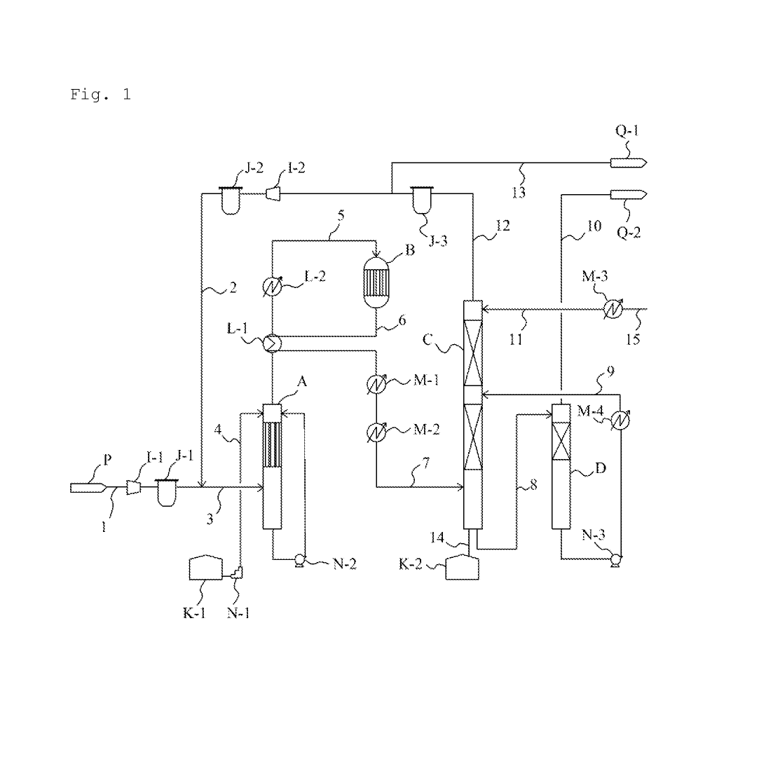

FIG. 1 is a schematic flow diagram illustrating a production process according to an embodiment of the present invention. In particular, FIG. 1 is a schematic flow diagram illustrating a process that is intended to give an aldehyde as a main target.

In the embodiment illustrated in FIG. 1, hydrogen gas is fed from a hydrogen installation P via a line 1, compressed with a compressor I-1, fed through a buffer tank J-1, merged with a recycled gas from a line 2, and charged via a line 3 into an evaporator A (carboxylic acid evaporator). A carboxylic acid is fed from a carboxylic acid tank K-1 using a pump N-1 via a line 4 to the evaporator A. The resulting vaporized carboxylic acid is, together with the hydrogen gas, heated with heat exchangers (heaters) L-1 and L-2 and charged via a line 5 into a reactor B, where the reactor B is packed with the catalyst according to the embodiment of the present invention. The evaporator A is equipped with a circulating pump N-2. The carboxylic acid is hydrogenated in the reactor B to give a main product aldehyde and/or alcohol, as well as non-condensable components such as methane, ethane, ethylene, and carbon dioxide, and condensable components such as ketones (e.g., acetone) and water. In addition, the hydrogenation also gives C2 or higher hydrocarbons, such as propylene, 1-butene, 1-pentene, and 1-hexene.

The carboxylic acid hydrogenation may be performed according to a known procedure. For example, the hydrogenation may be performed typically by allowing the carboxylic acid to react with hydrogen in the presence of the catalyst according to the embodiment of the present invention. The catalyst according to the embodiment of the present invention is preferably subjected to a reduction treatment before use in the carboxylic acid hydrogenation. The reduction treatment may be performed typically by contacting the catalyst with hydrogen. The reduction treatment may be performed typically by passing hydrogen (H₂) gas at a rate of 30 to 300 ml/min at a temperature of 50° C. to 500° C. and a pressure of 0.1 to 5 MPa.

The reaction in the reactor may be performed at a temperature of typically 250° C. to 400° C., and preferably 270° C. to 350° C. The reaction, if performed at an excessively high temperature, may readily cause by-productions of ketones such as acetone to be increased and cause the target product(s) to be produced with lower selectivity. The reaction in the reactor may be performed at normal atmospheric pressure, under reduced pressure, or under pressure (under a load) without limitation, but the reaction pressure is typically from 0 to 10 MPa, and preferably from 0.1 to 3 MP. The contact time in the reactor is typically 0.1 to 1 sec, and preferably 0.1 to 0.5 sec.

Hydrogen and the carboxylic acid may be fed to the reactor with a hydrogen to carboxylic acid ratio (mole ratio) of typically 0.5 to 50, and preferably 2 to 25.

The carboxylic acid in the reactor is preferably converted with a conversion of 50% or less (e.g., 5 to 50%). The hydrogenation reaction, if performed with a carboxylic acid conversion of greater than 80%, may cause by-products (e.g., ethyl acetate) to be more readily formed, and this may cause the aldehyde to be formed with a lower selectivity. To eliminate or minimize these, the residence time and the hydrogen flow rate in the reactor are preferably adjusted so that the conversion from the carboxylic acid becomes 80% or less.

The reaction between the carboxylic acid and hydrogen gives gaseous reaction products mainly including the unconverted carboxylic acid, unconverted hydrogen; and reaction products such as aldehyde, alcohol, water, and other products (e.g., carboxylic esters such as ethyl acetate; and ketones such as acetone), as described above.

The gaseous reaction products may be separated into non-condensable gases and condensable components, and the condensable components may be used as a reaction liquid (reaction mixture). The separation of the gaseous reaction products into non-condensable gases and condensable components may be performed typically, but not limitatively, via an absorption step. In the absorption step, a reaction fluid from the carboxylic acid hydrogenation is charged into an absorber (absorption column), and condensable components are absorbed from the reaction fluid with an absorbing liquid to separate the condensable components from the non-condensable gases. At least part of the by-produced C2 or higher hydrocarbons is absorbed with the absorbing liquid. In the production process according to the embodiment of the present invention, the condensable components absorbed by the absorbing liquid (a mixture of the condensable components and the absorbing liquid) are also included in the “reaction liquid”. In the absorption step, part of the non-condensable gases is dissolved in the absorbing liquid. However, hydrogen is efficiently separated from other non-condensable gas components by providing a stripping step. In the stripping step, a bottom liquid from the absorber is decompressed (reduced in pressure) to strip the dissolved non-condensable gases from the absorbing liquid, and the residual liquid after the non-condensable gas stripping is recycled to the absorber.

The absorption step may be performed typically in the following manner. The reaction fluid from the carboxylic acid hydrogenation is charged into the absorber. From the reaction fluid, the condensable components are absorbed with the absorbing liquid, and the non-condensable gases are dissolved in the absorbing liquid. The absorption step is generally performed by feeding the absorbing liquid and the reaction fluid from the reaction step to the absorber and contacting the two components with each other in the absorber. Examples of the absorber include, but are not limited to, publicly known or well-known gas absorbers with configurations typically of packed columns, plate columns, spray columns, and wetted wall columns.

In the stripping step, the bottom liquid from the absorber is reduced in pressure (decompressed) to strip the dissolved non-condensable gases from the absorbing liquid, and the residual liquid after the non-condensable gas stripping is recycled to the absorber. The stripping step is generally performed by feeding the bottom liquid of the absorber from the absorption step to a stripper under reduced pressure to strip the non-condensable gases. The bottom liquid herein is the absorbing liquid after absorption/dissolution of the condensable components and non-condensable gases. Examples of the stripper include, but are not limited to, publicly known or well-known gas strippers with configurations typically of packed columns, plate columns, spray columns, wetted wall columns, and gas-liquid separators.

In the embodiment illustrated in FIG. 1, the reaction fluid flown out from the reactor B is fed via a line 6 through the heat exchanger L-1, cooled with heat exchangers (coolers) M-1 and M-2, and charged via a line 7 into a lower portion of an absorber C. A recycled liquid as an absorbing liquid is charged via a line 9 into the absorber C. The “recycled liquid” herein refers to a bottom liquid from an after-mentioned stripper D. The recycled liquid mainly absorbs and dissolves therein hydrogen, methane, ethane, ethylene, and carbon dioxide, which are non-condensable gases. An upper-phase distillate may be charged as an absorber supply liquid via a line 11 into the absorber C, where the upper-phase distillate is rich in an azeotropic solvent that undergoes azeotropy with water. The “absorber supply liquid” refers to an absorbing liquid other than the recycled liquid. The absorber supply liquid absorbs, together with the non-condensable gases, an aldehyde which is a low-boiling condensable component. The upper-phase distillate is fed via a line 15 through a cooler M-3 to the line 11. The bottom liquid from the stripper D (recycled liquid; via the line 9) and the upper-phase distillate (absorber supply liquid; via the line 11) may be charged into the absorber C at any positions which may be selected as appropriate in consideration typically of absorption efficiency of the aldehyde and non-condensable gases. However, the recycled liquid and the absorber supply liquid may be charged respectively to an intermediate portion and an upper portion of the absorber C.

An absorber C bottom liquid is divided into a line 14 and a line 8. The line 14 leads to a reaction liquid tank K-2. The line 8 leads to the stripper D. The bottom liquid in the line 14 is stored as a reaction liquid in the reaction liquid tank K-2. Where necessary, the stored reaction liquid may be subjected to a purification step. At the stripper D, the bottom liquid fed via the line 8 is decompressed, and the dissolved non-condensable gases hydrogen, methane, ethane, ethylene, and carbon dioxide are stripped from the absorbing liquid and sent via a line 10 to a vent Q-2. The residual liquid after the non-condensable gas stripping is recycled via the line 9 to the absorber C.

The absorbing liquid to be charged into the absorber C may include the absorber C bottom liquid (recycled liquid) alone. However, when the aldehyde is acetaldehyde which has a low boiling point of 20° C., the absorbing liquid preferably includes an absorbing liquid devoid of or approximately devoid of acetaldehyde. This is preferred for higher recovery of acetaldehyde. Preferred examples of the absorbing liquid herein include, but are not limited to, an azeotropic-solvent-containing liquid for use in separation of unreacted carboxylic acid and by-produced water from each other via azeotropic distillation; and aqueous solutions of the carboxylic acid, such as a residual liquid after the separation of the aldehyde from the absorber C bottom liquid.

Assume that the azeotropic-solvent-containing liquid is used as the absorbing liquid. In this case, the azeotropic-solvent-containing liquid may contain the azeotropic solvent in a content of typically 10% by weight or more, preferably 30% by weight or more, more preferably 50% by weight or more, and furthermore preferably 75% by weight or more.

The azeotropic solvent forms an azeotrope with water, where the azeotrope has a lower boiling point. In addition, the azeotropic solvent is separable from water thereafter. These allow the carboxylic acid and water to be separated from each other more easily. Examples of the azeotropic solvent include, but are not limited to, esters such as isopropyl formate, propyl formate, butyl formate, isoamyl formate, ethyl acetate, isopropyl acetate, propyl acetate, butyl acetate, methyl propionate, ethyl propionate, methyl butyrate, ethyl butyrate, and isopropyl butyrate; ketones such as methyl ethyl ketone, methyl propyl ketone, methyl isobutyl ketone, diethyl ketone, and ethyl propyl ketone; aliphatic hydrocarbons such as pentane, hexane, and heptane; alicyclic hydrocarbons such as cyclohexane, methylcyclohexane, and dimethylcyclohexane; and aromatic hydrocarbons such as benzene and toluene.

Among them, ethyl acetate is preferred as the azeotropic solvent, because the ethyl acetate is often present as a by-product of the hydrogenation of the carboxylic acid (in particular, acetic acid), and this may eliminate the need for the step of recovering the azeotropic solvent.

Esters having a boiling point of 100° C. to 118° C. at normal atmospheric pressure are also preferred as the azeotropic solvent. Examples of the esters of this type include, but are not limited to, propyl acetate (boiling point: 102° C.), isobutyl acetate (boiling point: 117° C.), sec-butyl acetate (boiling point: 112° C.), isopropyl propionate (boiling point: 110° C.), methyl butyrate (boiling point: 102° C.), and ethyl isobutyrate (boiling point: 110° C.) These esters are preferred for reasons as follows. The esters form, with water, azeotropes having high water proportions and having lower boiling points as compared with acetic acid. This allows easier separation of the carboxylic acid (in particular, acetic acid) and water from each other. In addition, the esters do not undergo azeotropy with the alcohol (in particular, ethanol) or form, if any, with the alcohol (in particular, ethanol), azeotropes having low proportion of the alcohol (in particular, ethanol). Thus, these esters, when used as the azeotropic solvent, are relatively easily separated and recovered as the azeotropic solvent.

Methane, which is often present as a non-condensable gas, is more readily dissolved in an azeotropic solvent having low polarity than in an aqueous acetic acid solution having high polarity. This makes the azeotropic solvent suitable as the absorbing liquid for absorbing non-condensable gases.

The ratio (weight ratio) in amount of the absorber supply liquid (line 11) to the reaction fluid (line 7) each fed to the absorber C is typically 0.1 to 10, and preferably 0.3 to 2. The ratio (weight ratio) in amount of the recycled liquid (line 9) to the reaction fluid (line 7) each fed to the absorber C is typically 0.05 to 20, and preferably 0.1 to 10.

The absorber C may include typically 1 to 20 theoretical trays and preferably 3 to 10 theoretical trays. In the absorber C, the temperature is typically 0° C. to 70° C., and the pressure is typically 0.1 to 5 MPa (absolute pressure).

In the stripper D, the temperature is typically 0° C. to 70° C.; and the pressure is not limited, as long as being lower as compared with the absorber C, but is typically 0.05 to 4.9 MPa (absolute pressure). The pressure of the stripper D may be lower than the pressure of the absorber C by typically 0.05 to 4.9 MPa, and preferably 0.5 to 2 MPa, while the difference in pressure may be selected as appropriate from the viewpoints typically of non-condensable gas stripping efficiency and aldehyde loss reduction.

In the production process according to the embodiment of the present invention, the selectively to the aldehyde is typically 30% to 90%, and preferably 40% to 90%, while the selectivity may vary also depending on the reaction conditions. The selectivity to the alcohol is typically 5% to 50%, and preferably 5% to 40%, while the selectivity may vary also depending on the reaction conditions. The selectivity to the total of the aldehyde and the alcohol is typically 60% or more, preferably 70% or more, and more preferably 80% or more. The aldehyde yield is typically 10% to 50%, and preferably 20% to 50%. The alcohol yield is typically 10% to 50%, and preferably 20% to 50%. The selectivities and yields of the aldehyde and the alcohol may be determined typically by analyzing the reaction liquid typically by gas chromatography.

The production process according to the embodiment of the present invention gives the aldehyde with a purity of typically 90.0% by weight or more, preferably 95.0% by weight or more, and furthermore preferably 98.0% by weight or more. The production process gives the alcohol with a purity of typically 90.0% by weight or more, preferably 95.0% by weight or more, and furthermore preferably 98.0% by weight or more. The resulting aldehyde and/or alcohol may be subjected to further purification typically via distillation according to necessity, so as to have a still higher purity.

The production process according to the embodiment of the present invention uses the catalyst according to the embodiment of the present invention and effectively restrains by-production of C2 or higher hydrocarbons. The selectivity to the C2 or higher hydrocarbons is typically 15% or less, and preferably 10% or less. By adjusting the reaction conditions as mentioned above, the production process selectively yields the target product(s). This also restrains formation of ketones such as acetone, and gases such as carbon dioxide, carbon monoxide, and methane, which are formed as a result of other side reactions. The selectivity to ketones such as acetone is typically 10% or less, and preferably 5% or less. The selectivity to gases such as carbon dioxide, carbon monoxide, and methane is typically 10% or less, and preferably 5% or less.

### EXAMPLES

The present invention will be illustrated in further detail with reference to several examples below. It should be noted, however, that the examples are by no means intended to limit the scope of the present invention. The “content of palladium (Pd)” refers to a content in terms of palladium (Pd) element; and the “content of molybdenum (Mo)” refers to a content in terms of molybdenum (Mo) element. The “weight proportions of palladium (Pd) and molybdenum (Mo) in catalyst as charged” refer to amounts (weight proportions) of these elements per 100 parts by weight of the iron oxide (Fe₂O₃).

Catalyst Reactivity Evaluation 1

A sample catalyst (1.0 cc) was packed in a steel-use stainless (SUS) reaction tube having a diameter of 12 mm and being coupled to a fixed-bed gas phase continuous flow reactor. The reaction tube packed with the catalyst was heated for a pretreatment with an electric furnace in a hydrogen gas stream at 100 mL/min for 12 hours so that the catalyst bed temperature reached 300° C.

After the pretreatment, hydrogen gas (200 mL/min) and acetic acid liquid (0.103 cc/min) were circulated in the reactor for a reaction so that the contact time was 0.25 sec, and the hydrogen to acetic acid charge mole ratio (H₂/AC) was 5.0. Reactor outlet gases were cooled with a cooler to separate into gases and a liquid (condensed liquid), and the condensed liquid was trapped. Of the non-condensed gases, low-boiling components such as acetaldehyde were bubbled into 300 cc of water to be trapped. In addition, gas components that had not been trapped were trapped as gases. During the reaction, the electric furnace and a back pressure control valve were adjusted so that the catalyst bed temperature was 315° C. and the reaction pressure was 0.4 MPa. Twelve (12) hours or longer into the reaction, the condensed liquid after cooling with the cooler, the trapped liquid, and the trapped gases were collected for a predetermined time and were subjected to quantitative analyses and composition analyses.

The compositions were analyzed using a gas chromatograph and a Karl Fischer moisture meter.

### Comparative Example 1

### Preparation of Catalyst (A)

To 20 mL of water, 25.00 g of iron(III) nitrate hexahydrate (from Wako Pure Chemical Industries, Ltd.), 0.25 g of palladium(II) nitrate hydrate (from Johnson Matthey), and 1.07 g of citric acid (from Wako Pure Chemical Industries, Ltd.) were added, and stirred until they were dissolved to give a solution. The solution was dried to give a solid on a hot water bath at 80° C. until approximately no moisture remained. The resulting solid was further dried at 110° C. for 24 hours, fired at 400° C. for 5 hours, and yielded oxides. The oxides were compressed and compacted into tablets, crushed, classified through a 7-10 mesh, and yielded a catalyst (A).

The catalyst (A) contained 2.0 parts by weight of palladium (Pd).

### Example 1

### Preparation of Catalyst (B)

To 5 mL of water, 0.06 g of hexaammonium molybdate tetrahydrate (from Wako Pure Chemical Industries, Ltd.) was added and stirred until it was dissolved to give a solution. The oxides (5.00 g) prepared in Comparative Example 1 were immersed in the solution, and dried on a hot water bath at 80° C. until approximately no moisture remained, to give a solid. The solid was further dried at 110° C. for 24 hours, was fired at 400° C. for 5 hours, and yielded oxides. The oxides were compressed and compacted into tablets, crushed, classified through a 7-10 mesh, and yielded a catalyst (B).

The catalyst (B) contained 2.0 parts by weight of palladium (Pd) and 0.7 part by weight of molybdenum (Mo).

### Example 2

### Preparation of Catalyst (C)

A catalyst (C) was prepared in a similar manner to Example 1, except for using the hexaammonium molybdate tetrahydrate in an amount of 0.32 g.

The catalyst (C) contained 2.0 parts by weight of palladium (Pd) and 3.5 parts by weight of molybdenum (Mo).

### Example 3

### Preparation of Catalyst (D)

A catalyst (D) was prepared in a similar manner to Example 1, except for using the hexaammonium molybdate tetrahydrate in an amount of 0.64 g.

The catalyst (D) contained 2.0 parts by weight of palladium (Pd) and 7.0 parts by weight of molybdenum (Mo).

### Example 4

### Preparation of Catalyst (E)

A catalyst (E) was prepared in a similar manner to Example 1, except for using the hexaammonium molybdate tetrahydrate in an amount of 3.19 g.

The catalyst (E) contained 2.0 parts by weight of palladium (Pd) and 35 parts by weight of molybdenum (Mo).

The catalysts (A) to (E) prepared in Comparative Example 1 and Example 1 to 4 were subjected to the catalyst reactivity evaluation 1, and results of which are presented in Table 1.

TABLE 1

| | | | | | | | | | | | | Yield |
|---|---|---|---|---|---|---|---|---|---|---|---|---|
| | Weight proportion in | | Conversion | Selectivity (%) | | | | | | | | (%) |
| | catalyst as charged | | (%) | Acetal- | | | Carbon | Carbon | | Hydrocarbons | | Acetal- |
| | Mo | Pd | Acetic acid | dehyde | Ethanol | Acetone | dioxide | monoxide | Methane | (C2 or higher) | Others | dehyde |
| Example 1 | 0.7 | 2.0 | 40% | 69% | 15% | 2% | 1% | 1% | 3% | 8% | 2% | 28% |
| Example 2 | 3.5 | 2.0 | 44% | 60% | 18% | 7% | 4% | 1% | 1% | 5% | 4% | 26% |
| Example 3 | 7.0 | 2.0 | 42% | 56% | 19% | 10% | 6% | 0% | 1% | 4% | 4% | 23% |
| Example 4 | 35 | 2.0 | 33% | 62% | 15% | 8% | 5% | 1% | 1% | 3% | 3% | 20% |
| Comparative | — | 2.0 | 33% | 68% | 14% | 2% | 1% | 1% | 2% | 9% | 2% | 22% |
| Example 1 | | | | | | | | | | | | |

### Comparative Example 2

### Preparation of Catalyst (F)

A catalyst (F) was prepared in a similar manner to Comparative Example 1, except for using the palladium(II) nitrate hydrate in an amount of 1.26 g, and using the citric acid in an amount of 5.33 g.

The catalyst (F) contained 10 parts by weight of palladium (Pd).

### Example 5

### Preparation of Catalyst (G)

A catalyst (G) was prepared in a similar manner to Example 1, except for using the oxides prepared in Comparative Example 2 instead of the oxides prepared in Comparative Example 1; and using the hexaammonium molybdate tetrahydrate in an amount of 0.59 g.

The catalyst (G) contained 10 parts by weight of palladium (Pd) and 7.0 parts by weight of molybdenum (Mo).

The catalysts (F) and (G) prepared in Comparative Example 2 and Example 5 were subjected to the catalyst reactivity evaluation 1, and results of which are presented in Table 2.

TABLE 2

| | Weight | | | | | | | | | | | |
|---|---|---|---|---|---|---|---|---|---|---|---|---|
| | proportion in | | | | | | | | | | | |
| | catalyst as | | Conversion | Selectivity (%) | | | | | | | | |
| | charged | | (%) | Acetal- | | | Carbon | Carbon | | Hydrocarbons | | Yield (%) |
| | Mo | Pd | Acetic acid | dehyde | Ethanol | Acetone | dioxide | monoxide | Methane | (C2 or higher) | Others | Acetaldehyde |
| Example 5 | 7.0 | 10 | 62% | 40% | 34% | 9% | 5% | 1% | 1% | 1% | 9% | 25% |
| Comparative | — | 10 | 44% | 64% | 20% | 4% | 2% | 2% | 1% | 5% | 2% | 29% |
| Example 2 | | | | | | | | | | | | |

### Comparative Example 3

### Preparation of Catalyst (H)

A catalyst (H) was prepared in a similar manner to Comparative Example 1, except for using the palladium(II) nitrate hydrate in an amount of 2.51 g and using the citric acid in an amount of 10.66 g.

The catalyst (H) contained 20 parts by weight of palladium (Pd).

### Example 6

### Preparation of Catalyst (I)

A catalyst (I) was prepared in a similar manner to Example 1, except for using the oxides prepared in Comparative Example 3 instead of the oxides prepared in Comparative Example 1; and using the hexaammonium molybdate tetrahydrate in an amount of 0.54 g.

The catalyst (I) contained 20 parts by weight of palladium (Pd) and 7.0 parts by weight of molybdenum (Mo).

The catalysts (H) and (I) prepared in Comparative Example 3 and Example 6 were subjected to the catalyst reactivity evaluation 1, and results of which are presented in Table 3.

TABLE 3

| | Weight | | | | | | | | | | | |
|---|---|---|---|---|---|---|---|---|---|---|---|---|
| | proportion | | | | | | | | | | | |
| | in catalyst | | Conversion | Selectivity (%) | | | | | | | | |
| | as charged | | (%) | Acetal- | | | Carbon | Carbon | | Hydrocarbons | | Yield (%) |
| | Mo | Pd | Acetic acid | dehyde | Ethanol | Acetone | dioxide | monoxide | Methane | (C2 or higher) | Others | Acetaldehyde |
| Example 6 | 7.0 | 20 | 63% | 38% | 32% | 10% | 6% | 1% | 1% | 1% | 11% | 24% |
| Comparative | — | 20 | 46% | 56% | 23% | 6% | 3% | 1% | 1% | 7% | 3% | 26% |
| Example 3 | | | | | | | | | | | | |

Catalyst Reactivity Evaluation 2

A sample catalyst (1.0 cc) was packed in a steel-use stainless (SUS) reaction tube having a diameter of 12 mm and being coupled to a fixed-bed gas phase continuous flow reactor. The reaction tube packed with the catalyst was heated for a pretreatment in a hydrogen gas stream at 100 mL/min for 12 hours using an electric furnace so that the catalyst bed temperature reached 300° C.

After the pretreatment, reactions were performed under reaction conditions of different temperatures, pressures, contact times (durations), hydrogen to acetic acid mole ratios (H₂/AC) given in Tables 4 and 5 below. Reactor outlet gases were cooled with a cooler to separate into gases and a liquid (condensed liquid), and the condensed liquid was trapped. Of non-condensed gases, low-boiling components such as acetaldehyde were bubbled into 300 cc of water to be trapped. In addition, gas components that had not been trapped were trapped in a gaseous state. Twelve (12) hours or longer into the reaction, the condensed liquid after cooling with the cooler, trapped liquid, and trapped gases were collected for a predetermined time and were subjected to quantitative analyses and composition analyses.

The compositions were analyzed using a gas chromatograph and a Karl Fischer moisture meter.

The catalyst (G) prepared in Example 5 was subjected to the catalyst reactivity evaluation 2, and the results of which are summarized in Table 4.

TABLE 4

| | | | | Con- | | | | | | | | | | |
|---|---|---|---|---|---|---|---|---|---|---|---|---|---|---|
| Reaction conditions | | | | version | Selectivity (%) | | | | | | | | | Yield |
| Temper- | | Contact | | (%) | | | | | Carbon | | | | Acetal- | (%) |
| ature | Pressure | time | H₂/ | Acetic | Acetal- | | | Carbon | mon- | Meth- | Hydrocarbons | | dehyde + | Acetal- |
| (° C.) | (MPa) | (sec) | AC | acid | dehyde | Ethanol | Acetone | dioxide | oxide | ane | (C2 or higher) | Others | ethanol | dehyde |
| 315 | 0.4 | 0.25 | 5 | 62% | 40% | 34% | 9% | 5% | 1% | 1% | 1% | 9% | 74% | 25% |
| 315 | 0 | 0.25 | 5 | 46% | 62% | 13% | 14% | 8% | 1% | 0% | 0% | 2% | 75% | 28% |
| 300 | 0 | 0.25 | 7 | 48% | 56% | 9% | 20% | 12% | 1% | 0% | 0% | 2% | 65% | 27% |
| 300 | 0.4 | 0.125 | 7 | 46% | 52% | 36% | 4% | 3% | 0% | 0% | 1% | 5% | 87% | 24% |

### Comparative Example 4

### Preparation of Catalyst (J)

A catalyst (J) was prepared in a similar manner to Example 1, except for using the oxides prepared in Comparative Example 3 instead of the oxides prepared in Comparative Example 1; and further using 11.58 g of silicon dioxide (Aerosil 200 from Nippon Aerosil Co., Ltd.).

The catalyst (J) contained 20 parts by weight of palladium (Pd) and 200 parts by weight of silicon dioxide (SiO₂)

### Example 7

### Preparation of Catalyst (K)

A catalyst (K) was prepared in a similar manner to Example 1, except for using the oxides prepared in Comparative Example 4 instead of the oxides prepared in Comparative Example 1; and using the hexaammonium molybdate tetrahydrate in an amount of 0.30 g.

The catalyst (K) contained 20 parts by weight of palladium (Pd), 7.0 parts by weight of molybdenum (Mo), and 200 parts by weight of silicon dioxide (SiO₂).

The catalysts (J) and (K) prepared in Comparative Example 4 and Example 7 were subjected to the catalyst reactivity evaluation 2, and the results of which are summarized in Table 5.

TABLE 5

| | Reaction conditions | | | | | | | |
|---|---|---|---|---|---|---|---|---|
| | | | Contact | | Conversion | | | |
| | Temperature | Pressure | time | | (%) | Selectivity (%) | | |
| | (° C.) | (MPa) | (sec) | H₂/AC | Acetic acid | Acetaldehyde | Ethanol | Acetone |
| Comparative | 315 | 0.4 | 0.25 | 5 | 15% | 77% | 8% | 2% |
| Example 4 | | | | | | | | |
| Example 7 | 315 | 0.4 | 0.25 | 5 | 20% | 75% | 8% | 2% |
| | 315 | 0.4 | 0.25 | 14 | 45% | 68% | 17% | 1% |

| | Selectivity (%) | | | | | | |
|---|---|---|---|---|---|---|---|
| | Carbon | Carbon | | Hydrocarbons | | Acetaldehyde + | Yield (%) |
| | dioxide | monoxide | Methane | (C2 or higher) | Others | ethanol | Acetaldehyde |
| Comparative | 1% | 2% | 1% | 7% | 3% | 85% | 11% |
| Example 4 | | | | | | | |
| Example 7 | 2% | 1% | 1% | 5% | 6% | 83% | 15% |
| | 1% | 1% | 1% | 4% | 7% | 84% | 30% |

Catalyst Property Evaluation

The oxides prepared in Example 5, Comparative Example 1, and Comparative Example 2 were shaped into cylindrical pellets having a diameter of 5 mm and a height of 5 mm. An aliquot (3.0 cc) of them was charged into a SUS reaction tube having a diameter of 25 mm and being coupled to a fixed-bed gas phase continuous flow reactor, and heated for a pretreatment in a hydrogen gas stream at 100 mL/min for 12 hours using an electric furnace so that the catalyst bed temperature reached 300° C.

After the pretreatment, a reaction was performed while allowing hydrogen gas (37.8 L/hr) and acetic acid liquid (0.232 cc/min) to pass through the reactor at a contact time of 0.25 sec with a hydrogen to acetic acid charge mole ratio (H₂/AC) of 7.0. During the reaction, the electric furnace and a back pressure control valve were adjusted so that the catalyst bed temperature was 315° C. and the reaction pressure was 0.4 MPa. The reaction was continued for 400 hours in Example 5 and Comparative Example 1, and for 200 hours in Comparative Example 2, and the catalyst crushing strengths and the compositions of the catalysts were measured before and after the reaction.

The crushing strength was determined by applying force to the flat portion of a sample cylindrical pellet having a diameter of 5 mm and a height of 5 mm using the digital hardness meter KHT-40N (Fujiwara Scientific Co., Ltd.) and measuring the strength at the time when the pellet collapsed.

The compositions were analyzed using an X-ray fluorescence analyzer and the organic elemental analyzer CHN Corder MT-5 (Yanaco New Science Inc.).

The evaluation results are summarized in Table 6.

TABLE 6
Catalyst Property Evaluation (reaction conditions: at 315° C. and 0.4 MPa
for a contact time of 0.25 sec with H₂/AC of 7.0 for a reaction time of 400 hr*)

| | Weight proportion in | | | Catalyst composition in concentration | | | | | Crushing |
|---|---|---|---|---|---|---|---|---|---|
| | catalyst as charged | | | (by weight) | | | | | strength |
| | Mo | Pd | Condition | Mo | Pd | Fe | O | C | (N) |
| Example 5 | 7.0 | 10 | Before | 6.3% | 8.1% | 62% | 23% | 0% | 157 |
| | | | reaction | | | | | | |
| | | | After | 6.6% | 8.4% | 65% | 18% | 1.3% | 111 |
| | | | reaction | | | | | | |
| Comparative | — | 2.0 | Before | — | 2.0% | 72% | 23% | 2% | 165 |
| Example 1 | | | reaction | | | | | | |
| | | | After | — | 1.6% | 71% | 9.4% | 12% | 2 |
| | | | reaction | | | | | | |
| Comparative | — | 10 | Before | — | 10% | 63% | 27% | 0% | 121 |
| Example 2 | | | reaction | | | | | | |
| | | | After | — | 11% | 80% | 1.4% | 7.6% | 2 |
| | | | reaction | | | | | | |

*The reaction time was 200 hr for Comparative Example 2 alone.

Tables 1 to 3 demonstrate that the catalysts according to the embodiment of the present invention offered lower selectivities to C2 or higher hydrocarbons. Tables 4 and 5 demonstrate that the catalysts according to the embodiment of the present invention, when used under controlled reaction conditions, offered controlled selectivities to aldehydes and/or alcohols. Table 6 demonstrates that the catalyst according to Example 5 less suffered from increase in carbon (C) concentration after the reaction, whereas the catalysts according to Comparative Example 1 and Comparative Example 2 suffered from significant increase in carbon (C) concentration after the reaction. Table 6 also demonstrates that the catalyst according to Example 5 less suffered from deterioration in crushing strength in terms of catalyst pellets after the reaction, whereas the catalysts according to Comparative Example 1 and Comparative Example 2 suffered from significant deterioration in crushing strength in terms of catalyst pellets after the reaction. These demonstrate that the catalysts according to the embodiment of the present invention restrain coking and less suffer from collapse of the catalyst pellets and deterioration in strength.

### REFERENCE SIGNS LIST

A evaporator
B reactor
C absorber
D stripper
I-1 and I-2 compressor
J-1 to J-3 buffer tank
K-1 carboxylic acid tank
K-2 reaction liquid tank
L-1 and L-2 heater
M-1 to M-4 cooler
N-1 to N-3 pump (delivery pump)
P hydrogen installation (hydrogen cylinder)
Q-1 and Q-2 vent
1 to 15 line

1. A catalyst for producing at least one of an aldehyde and an alcohol from a carboxylic acid via hydrogenation, the catalyst comprising:
molybdenum;
palladium; and
an iron oxide.

2. The catalyst according to claim 1,
wherein the catalyst contains 0.1 to 40 parts by weight of molybdenum and 1 to 50 parts by weight of palladium per 100 parts by weight of the iron oxide.

3. The catalyst according to one of claims 1 and 2,
wherein the catalyst is obtained by:
preparing a dispersion or solution containing:
a component including the molybdenum;
a component including the palladium; and
an iron component;
drying the dispersion or solution to give a solid; and
firing the solid.

4. The catalyst according to claim 1,
wherein the catalyst is in a form of pellets.

5. A process for producing at least one of an aldehyde and an alcohol, the process comprising hydrogenating a carboxylic acid in a gas phase in the presence of the catalyst according to claim 1.

6. The process according to claim 5,
wherein acetic acid as the carboxylic acid is hydrogenated to give at least one of acetaldehyde as the aldehyde and ethanol as the alcohol.
